(19) United States
(12) Patent Application Publication
(10) Pub. No.: US 2026/0226395 A1
(43) Pub. Date: Aug. 6, 2026

(54) METHOD FOR TREATING LIGNOCELLULOSIC BIOMASS

(71) Applicant: IFP Energies Nouvelles, Rueil-Malmaison (FR)

(72) Inventors: Matthieu ARAGONES, Rueil-Malmaison Cedex (FR); Sophie COUDERC, Rueil-Malmaison Cedex (FR); Gilles FERSCHNEIDER, Rueil-Malmaison Cedex (FR)

(73) Assignee: IFP Energies Nouvelles, Rueil-Malmaison (FR)

(21) Appl. No.: 19/149,169

(22) PCT Filed: Jan. 10, 2024
(86) PCT No.: PCT/EP2024/050443

§ 371 Date: Jul. 18, 2025

(30) Foreign Application Priority Data
Jan. 18, 2023 (FR) FR2300445

(51) Int. Cl. C12M 1/00; C12P 7/10; C12P 7/14; C12P 19/02

(52) U.S. Cl. CPC C12M 43/02; C12M 21/12; C12M 45/06; C12M 45/09; C12M 47/02; C12P 7/10; C12P 7/14; C12P 19/02

(57) ABSTRACT

A process for the treatment of a lignocellulosic biomass comprises:
a) a stage of pretreatment (2) of biomass, in order to obtain a pretreated biomass (3),
b) a first stage of solid/liquid separation of all or part (3a) of the pretreated biomass (3) into a first solid fraction (7) and a first liquid fraction (6) comprising a mixture of sugars,
c) a stage of enzymatic hydrolysis (14) of the first solid fraction (7) of pretreated biomass, in order to obtain a hydrolysate (15) in the form of sugar(s),
d) a stage of purification (24) of said first liquid fraction by bringing said first liquid fraction (6) into contact with microorganisms consuming only the C6 sugars, in order to obtain a purified liquid fraction (26),
e) a second stage e) of solid/liquid separation (27) of the purified liquid fraction (26).

### TECHNICAL FIELD

The present invention relates to the treatment of sugary liquors, in particular “second-generation (2G)” sugary liquors, which can be obtained from lignocellulosic biomass.

These sugary liquors can be used to produce other products via a chemical or biochemical route (for example alcohols, such as ethanol or butanol, or other molecules, for example solvents, such as acetone, intermediate products used in the chemical industry, and the like), in particular as replacements for petrochemical derivatives.

### PRIOR ART

Lignocellulosic biomass represents one of the most abundant renewable resources on Earth. The substrates considered are very varied; they relate both to ligneous substrates, such as various woods (hardwoods and softwoods), coproducts resulting from agriculture (wheat straw, corn cobs, and the like) or from other industries, such as the food-processing industry, the paper industry, and the like.

Various types of processes exist for converting lignocellulosic biomass into sugary liquors, according to the type of biomass. In the case of sacchariferous plants (sugar beet, sugar cane) and amylaceous plants (corn and wheat), “first-generation (1G)” sugary liquors are obtained, for example by extraction operations.

In the case of biomass of agricultural, forestry or paper-making residues type, “second-generation (2G)” sugary liquors are obtained by a biochemical transformation process which generally comprises a pretreatment stage and a stage of enzymatic hydrolysis by an enzymatic cocktail. The pretreatment generally comprises a stage of impregnation with an acidic or basic or oxidizing liquor, then cooking of the impregnated biomass, which is optionally accompanied by steam explosion.

The sugary liquors resulting from the hydrolysis can subsequently be treated, for example by fermentation, in order to convert them into alcohol, and the process also comprises separation and/or purification stages.

These sugary liquors can also originate from a mixture of sugary liquors originating from different types of biomass.

Lignocellulosic biomass is composed of three main polymers: cellulose (35% to 50% by weight), which is a polysaccharide essentially consisting of hexoses; hemicellulose (20% to 30% by weight), which is a polysaccharide usually consisting essentially of pentoses; and lignin (15% to 25% by weight), which is a polymer of complex structure and of high molecular weight, composed of aromatic alcohols connected via ether bonds. These different molecules are responsible for the intrinsic properties of the plant wall and are organized in a complex tangle.

Among the three base polymers which make up lignocellulosic biomass, cellulose and hemicellulose are those which make possible the production of 2G sugary liquors.

Usually, hemicellulose is predominantly broken down into sugars during the pretreatment and cellulose is converted into glucose by the enzymatic hydrolysis. However, access to crude cellulose remains difficult for enzymes to access, hence the need for the abovementioned pretreatment. This pretreatment makes it possible to modify the physicochemical properties of the lignocellulosic biomass in order to improve the accessibility of the cellulose to the enzymes and its reactivity to enzymatic hydrolysis.

There exist many technologies which concern the invention for carrying out this pretreatment, which will be grouped together below under the generic term of “cooking”: acidic cooking, alkaline cooking, cooking by auto-hydrolysis, steam explosion, and “organosolv pulping” processes. The latter process concerns a pretreatment in the presence of one or more organic solvents and generally water. The solvent can be an alcohol (ethanol), an acid of acetic acid or formic acid type, or also acetone.

Various configurations are reported, for example, in the document “Production of Bioethanol from Lignocellulosic Materials via the Biochemical Pathway: A Review”, M. Balat, Energy Conversion and Management, 52 (2011), 858-875, or also in the document “Bioethanol Production from Agricultural Wastes: An Overview”, N. Sarkar, S. Kumar Ghosh, S. Bannerjee and K. Aikat, Renewable Energy, 37 (2012), 19-27.

One of the most effective pretreatments is steam explosion, in particular under acidic conditions, which makes possible virtually complete hydrolysis of hemicellulose and a significant improvement in the accessibility and the reactivity of cellulose to enzymes. This pretreatment can be preceded by other treatment(s).

Thus, the pretreatment can generally comprise three stages which are the liquor preparation, the impregnation of biomass with this liquor and the pretreatment of the impregnated biomass, for example by cooking, optionally coupled with steam explosion. The patent FR 3 075 203 describes a process with impregnation of biomass with an acidic liquor, followed by cooking and steam explosion of the impregnated biomass, with adjustment of the acidity of the acidic liquor and a recycling thereof. The patent FR 3 075 201 also describes a process of pretreatment of biomass by acid impregnation, then steam explosion, with in addition washing of the means for feeding the reactors and recycling of the aqueous washing liquors in the process.

The sugary liquors thus obtained from lignocellulosic biomass, in particular after its pretreatment or after enzymatic hydrolysis in the case of “2G” sugars, are provided in the form of a mixture of sugars in an aqueous phase, where “C5” sugars (that is to say, sugars containing 5 carbons), such as xylose and arabinose, and “C6” sugars (that is to say, sugars containing 6 carbons), such as glucose, mannose and galactose, are found.

In particular, in the case of sugary liquors of 2G type resulting from the pretreatment of lignocellulosic biomass, a proportion of C5 sugars which is markedly higher than that of the C6 sugars is observed in the sugary liquor for the majority of lignocellulosic biomasses. The term “sugary liquor” is understood to mean that sugars are released by the pretreatment and that they can be dissolved by mixing the pretreated biomass with water, for example.

In point of fact, there exist outlets for C5 sugary liquors, specifically (for example to convert xylose into xylitol).

To a lesser extent, the sugary liquors can also contain furfural or furfural derivatives, products of degradation of sugars, carboxylic acids, furan compounds, which are fermentation inhibitors, which is harmful when it is desired to continue the conversion of the sugary liquors into alcohols by fermentation using microorganisms of the yeasts or bacteria type.

For example, the publication “Removal of the Fermentation Inhibitor, Furfural, Using Activated Carbon in Cellulosic-Ethanol Production” by Kuang Zhang et al. (Industrial & Engineering Chemistry Research, 2011, 50, 14055-14060) demonstrates the inhibitory nature of furfural at a content of 4 g/l for the bacterium Zymomonas mobilis A3 during fermentations.

There thus exists a need to separate or purify C5 sugars from other sugars, in particular C6 sugars, and optionally from other undesirable compounds, in order to obtain pure C5 sugary liquors, or at the very least sugary liquors with an enhanced content of C5 sugars with respect to other sugars in the sugary liquor, and this very particularly in the context of production of sugars or of alcohol from 2G lignocellulosic biomass.

Various purification/separation techniques have already been envisaged.

Thus, the patent application WO 2022/023686 describes a biomass treatment comprising a pretreatment, then enzymatic hydrolysis in order to obtain a sugary liquor comprising glucose, which is subsequently clarified, then purified by filtration on activated carbon, in order to remove suspended matter and to capture certain soluble contaminants. This is a simple technique but one which does not make it possible to separate C5 sugars from C6 sugars.

The publication “Removal of Furfural and HMF from Monosaccharides by Nanofiltration and Reverse Osmosis Membranes” by Tielin Wang et al. (Journal of the Energy Institute, 91(2018), 473-480) proposes to remove furfural and its derivative from a sugary liquor, in order to increase the yield of the fermentation of the sugary liquor, by techniques of nanofiltration and of reverse osmosis membranes. This is a complex and expensive technique, and one which does not make it possible to separate the C5 sugars both from the C6 sugars and from the furfural derivatives, except by multiplying the membrane filtration operations.

The patent application US 2013/0149761 proposes to bring about the precipitation of the non-sugar compounds using barium or calcium hydroxide, in order to obtain a liquor which contains only sugars of the glucose, xylose and other sugars type. This technique thus does not make it possible to selectively separate the sugars, but simply to remove, from the sugary liquor, minor compounds which are byproducts of the pretreatment of biomass.

The patent EP 3 990 464 describes the separation of glucose, from a mixture of C5 sugars and of C6 sugars, by adsorption of glucose on a zeolitic adsorbent of FAU type. This is an advantageous method, which does not, however, make possible optimum separation of glucose, which exhibits a selectivity which can be improved.

The patent EP 3 990 668 describes the separation of xylose from a mixture of C5 sugars and of C6 sugars, also by adsorption, in this instance of xylose, on a zeolitic adsorbent of FAU type, with the same limit in terms of selectivity in the separation carried out.

In point of fact, in one case (separation of glucose) as in the other (separation of xylose), it may be necessary, according to the applications targeted, to obtain extremely pure separated sugars.

An aim of the invention is thus to overcome the abovementioned disadvantages. An aim of the invention is to improve the techniques for the selective separation of sugars in a sugary liquor comprising different sugars, in particular C5 sugars and C6 sugars, by targeting in particular that liquors enriched in C5 sugars be obtained (“enriched” liquor is understood to mean that the sugary liquor obtained by the invention has a ratio of C5/C6 sugars which is greater than the ratio of the sugary liquor obtained without the invention).

Another aim of the invention is to incorporate this separation in a line for the production of sugar or of alcohol from lignocellulosic biomass, in order to improve the operation or the performance qualities thereof, in particular so as to obtain a higher quality of sugary liquor in the case of sugar production and/or so as to obtain a higher degree of conversion into alcohol in the case of alcohol production.

### SUMMARY OF THE INVENTION

A subject matter of the invention is first of all a process for the treatment of a lignocellulosic biomass, said process comprising:
a) a stage of pretreatment of biomass, comprising a cooking of biomass, optionally accompanied by steam explosion, in order to obtain a pretreated biomass,
b) a first stage of solid/liquid separation of all or part (3a) of the pretreated biomass (3) obtained in stage a) into a first solid fraction (7) of pretreated biomass and a first liquid fraction (6) comprising a mixture of compounds, in particular in the aqueous phase, comprising C5 sugars with 5 carbons and C6 sugars with 6 carbons, and the content by weight of C5 sugars of which is greater than the content by weight of C6 sugars,
c) a stage of enzymatic hydrolysis (14) of the first solid fraction (7) of pretreated biomass obtained in stage b), in order to obtain a hydrolysate (15) in the form of sugar(s), including C6 sugars with 6 carbons,
d) a stage of purification of said first liquid fraction by bringing said first liquid fraction into contact with first microorganisms consuming, among the sugars of said fraction, essentially only the C6 sugars, in order to obtain a purified liquid fraction containing C5 sugars and depleted in C6 sugars,
e) a second stage of solid/liquid separation of the purified liquid fraction containing C5 sugars and depleted in C6 sugars obtained in stage d) in order to obtain a second solid fraction comprising the first microorganisms and a second liquid fraction containing C5 sugars and depleted in C6 sugars.

The term “essentially only C6 sugars” is understood to mean, in the present text, the fact that the microorganism does not consume the other sugar(s), in particular the C5 sugars, which are present in the mixture, or else in a proportion so low that it is negligible/not measurable.

For the sake of brevity, in the present text, the sugars with 5 carbons will be denoted C5 sugars and the sugars with 6 carbons will be denoted C6 sugars.

Preferably, the first liquid fraction contains sugars resulting predominantly, in particular completely, from the depolymerization of the hemicelluloses present in the lignocellulosic biomass. This first fraction is a sugary liquor obtained without the addition of enzymes and obtained on conclusion of the pretreatment of a lignocellulosic biomass.

The invention has thus developed a novel technique for the separation of C5 and C6 sugars in a sugary liquor resulting from the pretreatment of lignocellulosic biomass: the invention has selected, in the “purification” stage d), a microorganism which will selectively consume the sugar, in this instance the C6 sugar, which it is desired to remove from the first liquid fraction which is a sugary liquor, in order to obtain a liquid fraction said to be purified in C5 sugars, which can be upgraded as is in the chemical industry in particular, or which can be used in a line for the production of alcohol, in particular of ethanol.

This is a separation by a biochemical route, rather than by a physical or chemical route, which is highly advantageous: it is both more simple to carry out than known filtration techniques and markedly more effective and selective.

Within the meaning of the invention, the microorganism can be a single type of microorganism or a combination of different microorganisms.

Within the meaning of the invention, the term “mixture in an aqueous phase of compounds comprising C5 sugars with 5 carbons and C6 sugars with 6 carbons” can also be denoted, for the sake of brevity, under the term of “sugary liquor”.

Within the meaning of the invention, the term “sugars” is understood to mean sugars in their monomeric form (and not in an oligomeric/polymeric form).

Within the meaning of the invention, “liquor” is equivalent to a mixture of compounds in an aqueous phase: the liquor is aqueous; it is the same for the first and second liquid fractions of the treatment process according to the invention described above.

In general, in the present text, unless expressly mentioned otherwise, all the liquid fractions are aqueous.

In this purification stage d), the microorganism will consume the C6 sugar of the first liquid fraction at least for its own maintenance/metabolism or growth: the C6 sugar can thus disappear, in its entirety or virtually in its entirety, by appropriately metering the amount/the activity of the microorganism as a function of the amount/concentration of C6 sugars of the first liquid fraction to be purified.

The microorganism can also, according to the amount of C6 sugars present in the liquid fraction, according to the operating conditions employed (for example pH, temperature, aeration, and the like) and according to the amount and the type of microorganism chosen, convert the sugar into another upgradable compound. (However, it should be noted that the microorganism can also consume a very low amount of C5 sugar, such as xylose, but it remains essentially a consumer of C6 sugars.)

Thus, advantageously, the microorganism can convert at least a part, in particular the majority, of the C6 sugars into alcohol, in particular into ethanol. The conversion into alcohol is not necessarily complete because the microorganism can use a small amount of sugars for nonalcohol byproducts or for its metabolism. The term “the majority” is thus understood here as meaning the fact that the microorganism can convert at least half of the C6 sugars into ethanol (in particular more than half, for example at least two thirds, of said C6 sugars, indeed even virtually their entirety).

In point of fact, ethanol is a highly upgradable product: it will be possible to easily separate it from the purified first liquid fraction. It is possible, for example, to separate the ethanol from an aqueous mixture by distillation or by entrainment by stripping. It is also possible to retain it, in all or part, as a mixture with the C5 sugars, in particular in order to make use of its antimicrobial properties.

The microorganism used in the purification stage d), in particular when it is a yeast, is in solid suspension in the separated first liquid fraction. In the majority of cases, thus, provision is made to separate these solid microorganisms, in order to be able to upgrade the purified sugary liquor devoid of solid suspension. This separation, provided in stage e) of solid/liquid separation of the process according to the invention, can use any device, known to a person skilled in the art, such as a filtration, centrifugation, settling, sieve or cyclone device, or the combination of several of these devices, for example.

It might be optional in the case where the purified liquid fraction is used, as is or after separation of the alcohol which it contains, for example for propagation of microorganisms of yeast type or fermentation.

The invention will thus “insert”, into a process for the treatment of biomass targeted at converting it into sugars and then optionally into alcohol, and which provides, in a known way, a pretreatment of biomass, then its enzymatic hydrolysis and then its optional fermentation, a stage of purification of the sugary liquor obtained at the end of pretreatment, which opens the route to a host of new possibilities of upgrading, of product quality and of yield of conversion of biomass, as described in detail later.

Advantageously, only a first part of the pretreated biomass obtained in stage a) is conveyed to the first stage b) of solid/liquid separation, and a second part (or the remainder) of the pretreated biomass is conveyed directly to the enzymatic hydrolysis stage c). The enzymatic hydrolysis can then be carried out on the two types of stream at the same time, one coming directly from the pretreatment, the other having passed through a solid/liquid separation stage, the relative proportion of the stream resulting from the impregnated biomass which goes to the solid/liquid separation and of the stream resulting from the impregnated biomass which goes directly to the enzymatic hydrolysis being adjustable, for example between a relative proportion from 90/10 to 10/90 by weight.

The enzymatic hydrolysis can thus be carried out jointly both
on the solid residue resulting from the solid/liquid separation b), because this residue still contains compounds which can be converted into sugars by enzymatic hydrolysis,
and on the pretreated biomass resulting directly from the pretreatment,
   which makes it possible to maintain a high yield of conversion by enzymatic hydrolysis with respect to the initial amount of biomass, despite the withdrawal of a portion of pretreated biomass in order to recover the soluble sugars therefrom.

The solid/liquid separation stage b) can, according to a preferred embodiment, comprise a first stage of contacting between the pretreated lignocellulosic biomass and water, then a second filtration stage, and optionally a washing operation.

When the solid/liquid separation stage b) comprises a stage of contacting the pretreated biomass with a fluid and then a washing stage, this can be carried out cocurrentwise or countercurrentwise, using in particular a part of the recovered liquid fraction as washing and/or contacting fluid.

The solid/liquid separation stage b) can, according to a preferred embodiment, be carried out on a filtration unit, in particular a belt filter or a filter press, by centrifugation or by settling.

The process according to the invention can also comprise a stage f) of fermentation by third microorganisms of the hydrolysate in the form of sugar(s) obtained in stage c), in order to obtain a fermented biomass comprising at least one alcohol, in particular ethanol. This is then a process for the production of alcohol of ethanol type which can act as biofuel.

Also, in this case, according to a preferred embodiment, the enzymatic hydrolysis stage b) and the fermentation stage f) can be carried out simultaneously. This is then known as SSCF (Simultaneous Saccharification and Co-Fermentation).

Preferably, at least a part of the second solid fraction comprising the first microorganisms which are obtained in stage e) is recycled in the purification stage d). This is because the microorganisms retain a certain activity for a certain time for a certain concentration of sugars, in this instance of C6 sugars, which activity goes beyond a single production when the purification takes place, for example non-continuously, in batch in a dedicated reactor: it is thus possible to recover the microorganisms from the purified liquid fraction and to reintroduce them into the reactor where the purification takes places, for the following n productions with optional contribution of supplementary microorganisms if necessary, as their activity decreases.

Advantageously, the second liquid fraction containing C5 sugars and depleted in C6 sugars which is obtained in stage e) contains alcohol, in particular ethanol, obtained by conversion of all or part of the C6 sugars under the action of the first microorganisms, as seen above. It is then possible, according to the invention, to separate, in a stage g), in particular by evaporation or stripping, said second liquid fraction into a third liquid fraction enriched in sugars and a fourth liquid fraction enriched in alcohol. This is because this separation between sugar(s) and alcohol can present various advantages according to the product/products which it is desired to upgrade.

Thus, if it is desired to upgrade a liquid fraction very pure in C5 sugars, it may be necessary, in order to observe a given degree of purity, to remove therefrom the alcohol which it contains. It is also possible to remove therefrom only a part, in order for a residual content of alcohol in the solution of sugars to guarantee an antimicrobial effect for it.

Furthermore, ethanol is per se an upgradable product, in two ways: either directly as upgradable product or by reincorporating it in the process of the invention, if it targets a conversion of biomass into alcohol (ethanol): it is thus possible, for example, to add it to the ethanol obtained in the process by enzymatic hydrolysis/fermentation, then distillation (in order to dehydrate it): it is possible to mix the two ethanol streams, either upstream or downstream of the dehydration device of distillation column type of the installation. It is thus possible to increase the yield of conversion of biomass into ethanol of the process.

Advantageously, it is also possible to send all or part of the fourth liquid fraction containing the alcohol obtained in stage g) to the fermentation stage f) or, as described in detail above, to mix all or part of said fourth liquid fraction with the fermented biomass comprising at least one alcohol obtained on conclusion of the fermentation stage f).

The process according to the invention can also comprise a stage h) of production of enzymes from second microorganisms, in particular fungi, in order to use said enzymes to ensure the enzymatic hydrolysis of stage c). Also, in this case, it is possible to send all or part of the second liquid fraction containing C5 sugars and depleted in C6 sugars which is obtained in stage e) or of the third liquid fraction enriched in sugars which is obtained in stage g) to said stage h) as substrate for growth of the second microorganisms and/or for production of enzymes by said microorganisms. This is the scenario when the process incorporates in situ production of enzymes in order to carry out the enzymatic hydrolysis of the pretreated biomass (the other alternative being to bring, onto the production site, enzymes produced on another site). Also, in this case, the liquid fractions (in all or part) which are obtained according to the invention as a result of the introduction of the purification stage, and which contain C5 sugars, are particularly appropriate for the growth of fungi and for the production of enzymes.

It should be noted that it is possible, equally or alternatively, to send all or part of the hydrolysate obtained on conclusion of the enzymatic hydrolysis and containing sugars, in particular, glucose, to stage h) of production of enzyme as substrate for growth of the second microorganisms, or for production of enzymes by the latter.

The process according to the invention can also comprise a stage i) of propagation of third fermentation microorganisms, in order to use said microorganisms, in particular yeasts, to ensure the fermentation of stage f) when it is provided. This is the scenario where the microorganisms are propagated in situ, on the site of treatment of biomass. Also, in this case, it is possible to send all or part of the second liquid fraction containing C5 sugars and depleted in C6 sugars which is obtained in stage e) or the third liquid fraction enriched in sugars which is obtained in stage g) to said stage i) as substrate for the propagation of said third microorganisms. Here again, these fractions (in all or part) are particularly appropriate for helping in the propagation of microorganisms of yeasts type used for the fermentation of hydrolyzed biomass, also called hydrolysate.

According to the process of the invention, it is also possible to send all or part of the second liquid fraction containing C5 sugars and depleted in C6 sugars which is obtained in stage e) or the third liquid fraction enriched in sugars which is obtained in stage g) to the fermentation stage f). In this case, when the production of alcohol is targeted, the C5 sugars contained in this/these fraction(s) continue their conversion into alcohol with the hydrolysate resulting from biomass.

It is thus seen that one, the other or both liquid fractions (or at least a part of one or the other of these fractions) containing C5 sugars and which are obtained as a result of the purification provided by the invention can be used according to at least three different modes, it being possible for each of these modes to be operated alternately or simultaneously with the other two. The choice will be made, in particular, according to whether the microorganisms for fermentation and production of enzymes are or are not produced in situ, according to whether the final product targeted is a solution of liquor of C5 sugars or of the alcohol (ethanol), indeed even both, and even according to the type of biomass, because the relative content of C6 sugars of the 1^{st} liquid fraction before purification can vary according to the biomass chosen.

The first liquid fraction obtained in stage b) can also comprise furfural. This is very particularly the case when the liquid fraction to be purified results from a biomass pretreated under acidic conditions. Also, the first microorganisms can advantageously also consume furfural, in particular at least in part by conversion of the furfural into alcohol, in particular into furfuryl alcohol. Here again, this consumption is highly advantageous, insofar as furfural is known to be an inhibitor with regard to fermentation reactions (using yeasts): the productivity and the yields of possible biochemical conversions expected for the purified liquor are thus improved. It has also been possible to observe that furfural can also be an inhibitor with regard to the growth of microorganisms producing enzymes, in particular of the fungus Trichoderma reesei.

The first liquid fraction obtained in stage b) can also comprise 5-(hydroxymethyl)furfural 5-HMF. This is very particularly the case when the liquid fraction to be purified results from a biomass pretreated under acidic conditions. Also, the first microorganisms can advantageously also consume 5-(hydroxymethyl)furfural, in particular at least in part by conversion of the 5-(hydroxymethyl)furfural into 5-(hydroxymethyl)furfuryl alcohol.

Here again, this consumption is highly advantageous, insofar as 5-HMF is also known to be an inhibitor with regard to fermentation reactions: the yields of possible biochemical conversions expected for the purified liquor are thus improved.

The first liquid fraction to be purified comprising C5 sugars with 5 carbons and C6 sugars with 6 carbons can also comprise other compounds of degradation of C5 or C6 sugars, including furan compounds, organic acids, high-molecular-weight compounds, in particular when it results from the pretreatment of lignocellulosic biomass at high temperature or by solvent.

What is advantageous, and surprising, in the process according to the invention is that the biochemical purification according to the invention of this first liquid fraction remains effective, even when the liquid fraction contains compounds of this type, whereas it might have been feared for them to potentially act as inhibitor(s) with regard to the conversion of C6 sugars during the purification according to the invention.

The first liquid fraction to be purified can also comprise inorganic salts.

The first liquid fraction to be purified can also comprise sugars in oligomeric form.

Preferably, the first and third microorganisms are chosen from yeasts, bacteria and fungi, and in particular from yeasts in the genus Saccharomyces, in particular the species Saccharomyces cerevisiae, or from bacteria in the genus Corynebacterium, in particular the species Corynebacterium, Zymomonas mobilis. The choice of Saccharomyces cerevisiae in particular is advantageous because the wild strain naturally consumes C6 sugars but not C5 sugars and studies have been carried out to genetically modify it in order for it to be precisely capable of consuming both types of sugars: the invention thus recommends returning to the genetically unmodified version of this yeast/the native version, in particular for the first microorganism, precisely to exploit its selectivity with regard to sugars, hitherto perceived as a disadvantage.

Preferably, the second microorganisms are chosen from fungi, in particular filamentous fungi, preferably those of the genus Trichoderma, in particular Trichoderma reesei.

According to a preferred embodiment of the invention, the pretreatment according to stage a) of biomass comprises:
a1) a substage of impregnation of biomass with a liquor, in particular an acidic liquor, in order to obtain an impregnated biomass (alternatively, the liquor can be basic or oxidizing or be only water),
a2) a substage of cooking the impregnated biomass, optionally accompanied by steam explosion, in order to obtain a pretreated biomass.

The process according to the invention can also comprise a stage j) of separation, in particular by distillation, of the fermented biomass obtained in stage f) in a form of alcohol, with an optional stage k) of solid/liquid separation of the fermented biomass before or after said separation stage j).

Stage j) makes it possible in particular to dehydrate the alcohol, and its preferred implementation uses a beer column to carry out a distillation, making it possible to recover, at the column top, the ethanol which it is desired to produce and, at the column bottom, vinasses.

The optional stage k) is preferably carried out before the separation stage j), in order to avoid any risk of traces of solid residue in the distillation column, when stage j) is carried out by distillation.

Preferably, the purification d) of the first liquid fraction comprising C5 sugars with 5 carbons and C6 sugars with 6 carbons with the microorganism is carried out at a temperature of between 20° C. and 60° C., in particular between 25° C. and 40° C., in particular between 30° C. and 35° C. The temperature of the treatment is to be adapted as a function of the microorganism chosen, each microorganism having a temperature range where its activity is at a maximum.

Advantageously, the purification d) of the first liquid fraction comprising C5 sugars with 5 carbons and C6 sugars with 6 carbons with the microorganism has a duration of between 5 minutes and 15 hours, in particular between 10 minutes and 8 hours.

Advantageously, the purification d) of the first liquid fraction comprising C5 sugars with 5 carbons and C6 sugars with 6 carbons with the microorganism can be carried out in batch mode, in fed-batch of said liquid fraction mode, or continuously.

The selected microorganism which will selectively consume the C6 sugar according to the invention, that is to say the first microorganism, can be added in one go directly with the first liquid fraction, or according to sequential feeding, independently or not of the feeding of the first liquid fraction.

The duration of the purification stage d) is to be adjusted as a function of the type and of the amount of microorganism used, as a function of the amount of C6 sugars in the liquid fraction to be purified, as a function of the amount of compounds said to be inhibitors of the microorganism (furan compounds, such as furfural or 5-(hydroxymethyl)furfural, carboxylic acids, such as acetic acid or formic acid, or phenolic compounds) for the yeast Saccharomyces cerevisiae, and as a function of the final content of C6 sugars acceptable in the purified liquor or, expressed otherwise, according to the degree of purity in C5 sugars sought.

Advantageously, the purification d) of the first liquid fraction comprising C5 sugars with 5 carbons and C6 sugars with 6 carbons with the microorganism can be carried out at a pH of between 2 and 10, in particular between 4 and 6, in particular between 4.5 and 5.5.

Advantageously, the purification d) of the first liquid fraction comprising C5 sugars with 5 carbons and C6 sugars with 6 carbons with the microorganism can be carried out under aerobic or anaerobic conditions, according to the microorganism chosen and the product desired (alcohol, growth of the microorganism, and the like) from the consumption of the sugars.

Advantageously, the concentration of microorganisms in the first liquid fraction comprising C5 sugars with 5 carbons and C6 sugars with 6 carbons is of between 1 and 200 g/kg of C6 sugars, in particular between 10 and 30 g/kg of C6 sugars.

The biomass from which the first liquid fraction comprising C5 sugars with 5 carbons and C6 sugars with 6 carbons results is of lignocellulosic type, originating in particular from forestry and/or agricultural and/or paper-making residues, and/or from sacchariferous plants and/or from amylaceous plants and/or from Fermentable Household Waste Fractions (FHWF).

The pretreatment of biomass from which the first liquid fraction is obtained can be a hydrolysis (without enzyme) or pretreatment of biomass comprising in particular impregnation of biomass with an acidic or basic or oxidizing liquor, followed by cooking of the impregnated biomass, in particular cooking with steam explosion.

The first liquid fraction comprising C5 sugars with 5 carbons and C6 sugars with 6 carbons is preferably obtained, according to the invention, after a solid/liquid separation stage b) preferably employing a biomass pretreated by steam explosion, optionally reslurried with a solvent (in particular water) and optionally washed with a solvent (in particular water).

Another subject matter of the invention is the purified first liquid fraction obtained in stage d) or the second liquid fraction obtained in stage e) according to the process of the invention (that is to say, the “purified liquor” according to the invention, before or after separation from the microorganisms by solid/liquid separation): the invention thus has as subject matter a liquid comprising a mixture of compounds in aqueous solution, which comprises C5 sugars and conversion products of C6 sugars and optionally other minority compounds present in the liquor to be purified.

For example, these conversion products of C6 sugars and other minority compounds are alcohols, such as ethanol, 5-(hydroxymethyl)furfuryl alcohol and furfuryl alcohol, in particular in the following concentrations;
C5 sugars: between 10 and 100 g/kg of solution, in particular between 20 and 60 g/kg of solution,
ethanol: between 1 and 12 g/kg of solution, in particular between 2 and 7 g/kg of solution,
5-(hydroxymethyl)furfuryl alcohol: between 0.1 and 9 g/kg of solution, in particular between 0.2 and 4 g/kg of solution,
furfuryl alcohol: between 0.05 and 9 g/kg of solution, in particular between 0.1 and 3.8 g/kg, in particular between 0.1 and 1.8 g/kg of solution.

The purified first liquid fraction obtained in stage d) or the second liquid fraction obtained in stage e) according to the process of the invention can thus contain alcohols, in particular an alcohol which results from the conversion of C6 sugars under the action of the microorganism, for example ethanol, butanol or isopropanol, and alcohols resulting in particular from the conversion of the two inhibitors (furfural and 5-HMF), also under the action of the microorganism.

The conversion products of the C6 sugars can also be microorganisms themselves, in particular yeasts, in the case where the treatment according to the invention is carried out under aerobic conditions. This is because C6 sugars are consumed by the microorganism for its own growth or its maintenance.

Advantageously, the purified liquid fraction obtained in stage d) or the second liquid fraction obtained in stage e) according to the process of the invention contains less than 0.5 g/kg of solution of C6 sugars, and preferably no C6 sugar, less than 0.1 g/kg of solution of furfural, in particular no furfural, and less than 0.1 g/kg of solution of 5-(hydroxymethyl)furfural, in particular no 5-(hydroxymethyl)furfural.

With the invention, it is thus possible to range as far as completely removing (by consumption or conversion) all the C6 sugar of the initial sugary liquor, and the same is true for the two furan derivatives: according to the constraints of degree of purity of the purified liquor in C5 sugars, the C6 sugar can thus be removed or at the very least kept at a very low content, at the level of an acceptable content of impurities, for example.

Advantageously, the purified liquid fraction obtained in stage d) or the second liquid fraction obtained in stage e) according to the process of the invention contains the following compounds:
glucose: between 0 and 0.3 g/kg of solution,
xylose: between 9 and 91 g/kg of solution,
arabinose: between 1 and 9 g/kg of solution,
galactose: between 0 and 0.1 g/kg of solution,
mannose: between 0 and 0.1 g/kg of solution,
5-(hydroxymethyl)furfuryl alcohol: between 0.1 and 9 g/kg of solution,
furfuryl alcohol: between 0.05 and 3.8 g/kg of solution,
ethanol: between 0 and 7 g/kg.

The first purified liquid fraction obtained in stage d) or the second liquid fraction obtained in stage e) according to the process of the invention can also contain carboxylic acids, such as acetic acid, formic acid or levulinic acid. Advantageously, the purified first liquid fraction obtained in stage d) or the second liquid fraction obtained in stage e) according to the process of the invention (“purified liquor” before or after separation from the microorganisms by solid/liquid separation) contains less than 8 g/kg of acetic acid, preferably less than 5 g/kg of acetic acid. The purified liquor according to the invention can also contain sugars in oligomeric form, for example compounds of arabinoxylan type, oligomers of pentoses and of hexoses. Advantageously, the purified liquor according to the invention contains between 0% and 100% of oligomeric sugars with respect to the monomeric sugars, preferably between 0.1% and 50%, in particular between 0.1% and 20%, of oligomeric sugars with respect to the monomeric sugars.

Another subject matter of the invention is the use of the purified liquor described above, which is thus a sugary liquor having a very high content of C5 sugars (with respect to its content of C6 sugars), for converting C5 sugars by a chemical route, in particular for converting xylose into xylitol, or by a biochemical route, in particular for acting as carbon-based substrate for the propagation of yeasts or for the induction of fungi for the production of enzymes.

Xylose is regarded as a platform molecule, and many upgrading pathways are possible (xylitol, monoethylene glycol, propanol, butanol, lactic acid, succinic acid, and the like).

Another subject matter of the invention is any installation for the treatment of a lignocellulosic biomass employing the biomass treatment process described above.

Another subject matter of the invention is an installation for the treatment of a lignocellulosic biomass, said installation comprising:
a) a unit for the pretreatment of biomass, comprising a device for cooking biomass, optionally accompanied by steam explosion, in order to obtain a pretreated biomass,
b) a first unit for solid/liquid separation (4), comprising in particular a filtration device, of all or part (3a) of the pretreated biomass (3) obtained in the unit a) into a first solid fraction (7) of pretreated biomass and a first liquid fraction (6) comprising a mixture of compounds, in particular in the aqueous phase, comprising C5 sugars with 5 carbons and C6 sugars with 6 carbons, and the content by weight of C5 sugars of which is greater than the content by weight of C6 sugars,
c) a unit for enzymatic hydrolysis (14) of the first solid fraction (7) of pretreated biomass obtained in stage b), in order to obtain a hydrolysate (15) in the form of sugar(s), including C6 sugars with 6 carbons,
a unit d) for purification of said first liquid fraction with said first liquid fraction being brought into contact with first microorganisms consuming, among the sugars of said fraction, essentially only the C6 sugars, in order to obtain a purified liquid fraction containing C5 sugars and depleted in C6 sugars,
a second unit e) for solid/liquid separation, comprising in particular at least one filtration or centrifugation device, of the purified liquid fraction containing C5 sugars and depleted in C6 sugars obtained in the unit d) in order to obtain a second solid fraction comprising the first microorganisms and a second liquid fraction containing C5 sugars and depleted in C6 sugars.

Advantageously, according to an alternative form, only a first part (3a) of the pretreated biomass (3) obtained in the pretreatment unit a) is conveyed to the first unit b) for solid/liquid separation, and a second part of the pretreated biomass (all the remainder of the stream of pretreated biomass, preferably) is conveyed directly to the enzymatic hydrolysis unit c).

The solid/liquid separation unit b) can, according to a preferred embodiment, comprise a first device for contacting between the pretreated lignocellulosic biomass and water, then a second filtration device, and optionally a washing device (the same device can be used with two functions, or two separate devices can be used).

Advantageously, in this installation, the second liquid fraction containing C5 sugars and depleted in C6 sugars which is obtained in the unit e) contains alcohol, in particular ethanol, obtained by conversion of all or part of the C6 sugars under the action of the first microorganisms, and said installation comprises a unit g) for separation of said second liquid fraction into a third liquid fraction enriched in sugars and a fourth liquid fraction enriched in alcohol, said separation unit g) comprising at least one evaporation or stripping device.

The invention will be described below in greater detail, with the help of nonlimiting examples and of the following figures:

### LIST OF THE FIGURES

FIG. 1 is a very diagrammatic representation of a reactor implementing the purification stage d) of the treatment process according to the invention, and representing the compounds of interest entering and leaving the reactor.

FIG. 2 is a graph representing, on the abscissa, the duration of the purification stage d) of the process according to the invention (in hours) and, on the ordinate, the change in the concentrations of compounds of a mixture of sugars which is treated according to the invention, in g/kg of reaction medium.

The abbreviations in FIG. 2 mean:
glu: glucose
xyl: xylose
ara: arabinose
gal: galactose
man: mannose

FIG. 3 is a very diagrammatic representation of a reactor implementing the purification stage d) of the treatment process according to the invention, and representing the compounds of interest entering and leaving the reactor under aerobic conditions, to propagate the microorganisms (yeasts).

FIG. 4 represents, in the form of a block diagram, a process for the treatment of lignocellulosic biomass with a view to converting it into alcohol (ethanol), without employing the purification according to the invention.

FIG. 5 represents, in the form of a block diagram, a process for the treatment of lignocellulosic biomass with a view to converting it into alcohol (ethanol), which modifies the process of FIG. 4 to employ the purification according to the invention.

FIG. 6 represents, in the form of a block diagram, a process for the treatment of lignocellulosic biomass with a view to converting it into sugar(s), without employing the purification according to the invention.

FIG. 7 represents, in the form of a block diagram, a process for the treatment of lignocellulosic biomass with a view to converting it into sugar(s), which modifies the process of FIG. 6 to employ the purification according to the invention.

FIG. 8 represents, in the form of a block diagram, a process for the treatment of lignocellulosic biomass with a view to converting it partly into sugar(s), partly into alcohol (ethanol), without employing the purification according to the invention.

FIG. 9 represents, in the form of a block diagram, a process for the treatment of lignocellulosic biomass with a view to converting it partly into sugar(s), partly into alcohol (ethanol), which modifies the process of FIG. 8 to employ the purification according to the invention.

FIG. 10 is a graph representing, on the abscissa, the time, expressed in hours, and, on the ordinate, along the left-hand vertical axis, the concentrations of furfural and 5 HMF, expressed in g/l, and, along the right-hand vertical axis, the concentration of ethanol in g/l, during the implementation of simultaneous enzymatic hydrolysis and fermentation (SSCF) of a pretreated biomass, with purification according to the invention.

FIG. 11 is a graph representing, on the abscissa, the concentration of ethanol (in % by weight) of the aqueous reaction medium obtained at the end of the treatment by enzymatic hydrolysis and fermentation of the pretreated biomass and, on the ordinate, the energy consumption (in MJ per kg of ethanol) to separate, by distillation, the ethanol from the aqueous phase starting from said aqueous reaction medium.

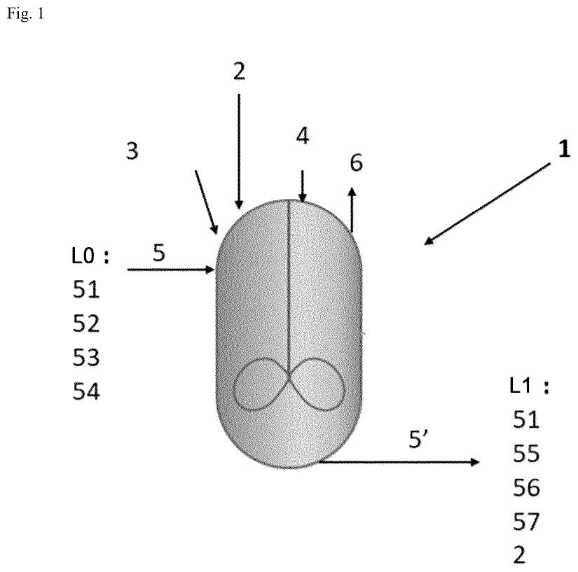

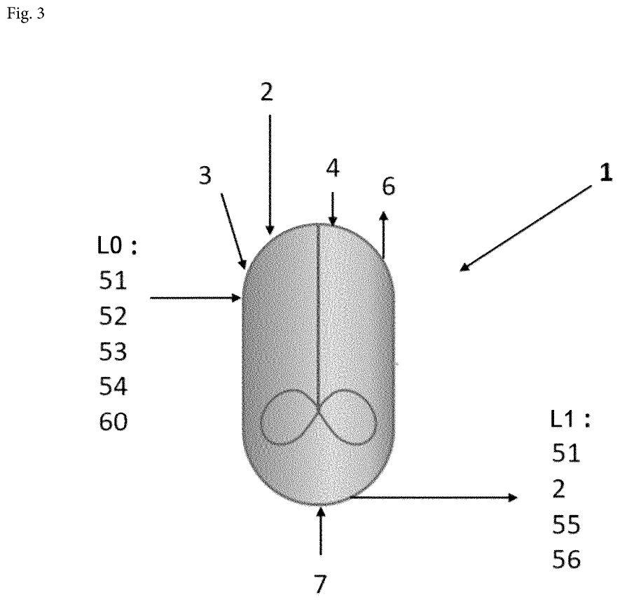

FIGS. 1 and 3 are extremely diagrammatic to make them easier to understand, and the reactor represented is thus a symbolic representation, which is not to scale, which does not foreshadow its dimensions and shape, and which does not show all the equipment necessary for its operation.

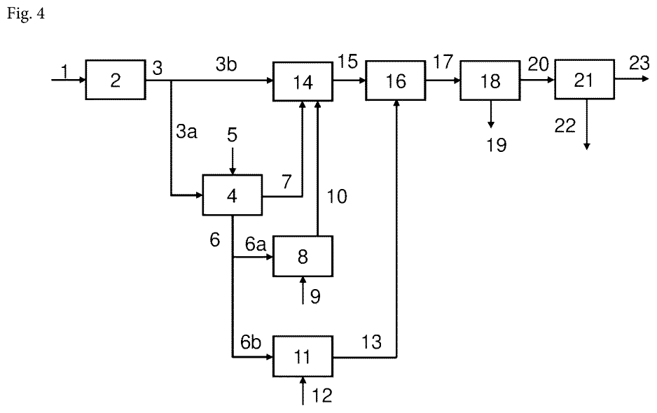

FIGS. 4 to 9, representing block diagrams of processes, are also highly simplified to make them easier to understand. They do not necessarily represent all the stages/items of equipment necessary for the processes described, concentrating on the most significant stages/items of equipment/streams in the light of the invention.

The references retain the same meaning from one figure to another.

### DESCRIPTION OF THE EMBODIMENTS

The invention incorporates, in a process for the conversion of biomass, of lignocellulosic type, into sugar(s) or into alcohol, a treatment for the biochemical purification of sugary liquors containing C5 sugars and, in a lower content/proportion, C6 sugars, so as to reduce, indeed even eliminate, the content of C6 sugars of these liquors and to thus obtain liquors “purified” in C5 sugars, in order to benefit therefrom, in particular either to increase the yield of conversion of biomass (more particularly into alcohol) or to obtain sugars of higher quality/purity.

Specifically, in the biomass treatment process, the invention is concerned with processes with pretreatment of biomass before enzymatic hydrolysis, which pretreatment results in the production of sugary liquors mixing C5 sugars and C6 sugars.

A description will thus be given:
to start with, and with the help of FIGS. 1 to 3, of the stage of purification of the sugary liquors,
then, subsequently, and with the help of the following figures, of the process for the conversion of lignocellulosic biomass producing these sugary liquors and incorporating this purification stage in order to benefit therefrom.

### Stage of Purification of the Sugary Liquors

This biochemical purification treatment is an operation of bringing the sugary liquor containing C5 and C6 sugars, which liquor is obtained by treatment of material based on lignocellulosic biomass and comprises predominantly C5 sugar, into contact with a microorganism which selectively consumes the C6 sugars: the C5 sugars are not modified and remain virtually intact in the liquor, whereas the C6 sugars will gradually be consumed by the microorganism for its own growth and/or to be converted into another compound, for example in the form of alcohol in the case of a fermentation.

For example, the native yeast Saccharomyces cerevisiae consumes the C6 sugars predominantly to multiply under aerobic conditions, and consumes the C6 sugars predominantly to produce ethanol under anaerobic conditions.

The operating conditions of the treatment are adapted as a function of the initial content of C6 sugars and of the final content of C6 sugars which is acceptable (no more C6 sugars at all, or a content less than a given value): temperature, pH, duration, aeration, choice and concentration of the microorganism.

### Examples of Purification of the Sugary Liquors

### Example 1 (Comparative)

The starting point is an initial sugary liquor L0 resulting from biomass pretreated by acid impregnation of biomass, then steam explosion, in accordance with the teaching of the abovementioned patent FR 3 083 126, to which reference will be made for further details.

Briefly, this pretreatment is an operation of impregnation of lignocellulosic biomass with an acidic liquor, followed by an operation of steam explosion of the impregnated biomass. At least a part of the pretreated biomass undergoes a liquid/solid separation operation on conclusion of the pretreatment stage, this liquid/solid separation stage comprising a stage of contacting between the pretreated lignocellulosic biomass and water, and a stage of filtration and optionally of washing. In order to obtain the liquor L0, 550 kg of pretreated biomass are mixed with 1010 kg of water, then filtered and then pressed. After filtration and pressing, 885 kg of a liquor L0, also known as hydrolysate C5, are obtained. As the pretreatment takes place under acidic conditions, this liquor has a pH of 2.

The concentrations of compounds of interest in this liquor L0 are given in table 1 below, with concentrations expressed in g per kg of liquor. It should be noted that it is possible for the composition to contain other impurities, the analysis of which has not been attempted, for example of the acid or (inorganic) salt type, in very low contents.

TABLE 1

| | Concentration | Liquor L0 | Type of sugar/compound |
|---|---|---|---|
| Xylose | g/kg | 34.4 | C5 sugar |
| Arabinose | g/kg | 3.5 | C5 sugar |
| Glucose | g/kg | 6.9 | C6 sugar |
| Galactose | g/kg | 1.1 | C6 sugar |
| Mannose | g/kg | 0.4 | C6 sugar |
| 5-HMF | g/kg | 0.4 | Potential fermentation inhibitor |
| | | | according to the concentration |
| Furfural | g/kg | 0.2 | Potential fermentation inhibitor |
| | | | according to the concentration |

It is seen that 82% of the sugars of the liquor L0 are C5 sugars, that 18% of the sugars are C6 sugars and that 5-HMF and furfural, which are known to inhibit biochemical reactions which can be envisaged as taking place on the sugars subsequently (for example for ethanolic fermentations with yeasts), are also present, in much lower contents than the sugars.

Sugary liquors resulting from pretreated biomass generally comprise a much higher amount of C5 sugars than that of C6 sugars, and there is a need, in order to upgrade them, to separate the C5 sugars from the C6 sugars. In point of fact, the techniques currently available are not very satisfactory (membrane filtrations in particular) because they are complex to carry out and generally do not make it possible to achieve, in the separated liquor of C5 sugars, the desired level of purity in C5 sugars.

For example, the publication “Removal of Furfural and HMF from Monosaccharides by Nanofiltration and Reverse Osmosis Membranes” by Tielin Wang et al. (Journal of the Energy Institute, 91(2018), 473-480) concludes that the most effective membranes for separating 5-HMF and furfural from a sugary liquor are the Desal-5 DK and Alfa Laval-NF membranes (less than 2% retention for 5-HMF and furfural) but that they result in too great a loss of sugars (from 2% to 8% for glucose and from 10% to 20% for xylose).

The publication “Removal of the Fermentation Inhibitor, Furfural, Using Activated Carbon in Cellulosic-Ethanol Production” by Kuang Zhang et al. (Industrial & Engineering Chemistry Research, 2011, 50, 14055-14060) concludes, for its part, that the commercial activated carbon Norit_1240 from Norit can selectively reduce the furfural concentration of a model solution of sugary liquor from 4 g/l to 0 g/l but C6 sugars (in this instance glucose) are not selectively separated from C5 sugars (in this instance xylose).

### Example 2 (According to the Invention)

The starting point is the sugary liquor L0 defined in example 1. Fermentation of the liquor L0 is carried out with a microorganism, the yeast Saccharomyces cerevisiae, in a native version which is not genetically modified and which exhibits the distinguishing feature of selectively consuming C6 sugars and not C5 sugars.

The yeast is inoculated at 0.5 g of yeasts per kg of solution, i.e. 60 g of yeasts per kg of C6 sugars. The experiment was carried out in a bioreactor at 33° C. and pH 5.3 for 6 hours. The treatment was carried out under anaerobic conditions in order to maximize the production of ethanol from C6 sugars by fermentation and not the production of biomass. This reaction can be carried out in batch mode, fed-batch mode or continuously. They can subsequently be separated in order to treat/upgrade them separately.

The reactions which result from the action of the yeast are shown diagrammatically in FIG. 1, which represents the bioreactor 1, with the following inputs:
2: the yeast,
3: a base (for regulating the pH and keeping the reaction medium under the conditions necessary for the activity of the chosen yeast), of NH₄OH, KOH or NaOH type, for example,
4: nutrients for the yeast,
5: the liquor L0, which contains, in the aqueous phase, C5 sugars 51, C6 sugars 52, 5-HMF 53 and furfural 54,
and with the exiting products:
6: emission of CO₂ in the gas phase, discharged at the top part of the bioreactor, evidence of the activity of the yeast,
5′: withdrawal of a liquor L1, resulting from the liquor L0 after the action of the yeast on the liquor L0, and which contains the yeast 2, C5 sugars 51, 5-(hydroxymethyl)furfuryl alcohol 55, furfuryl alcohol 56, ethanol 57 and yeasts 2.

The amounts of inputs (besides the concentrations indicated above) are specified below: In a stirred vessel, 985 kg of liquor L0 are mixed with:
0.4 kg of urea (for the nitrogen requirements),
0.5 kg of dry S. cerevisiae yeasts,
5 kg of yeast extract (nutrients),
9.1 kg of a 50% by weight KOH solution to adjust the pH to 5.3.

The concentrations of compounds of interest in the mixture in the initial state are given in table 2 below, with concentrations expressed in g per kg of liquor. It should be noted that it is possible for the composition to contain other impurities, the analysis of which has not been attempted, of the salt or acid type, in very low contents.

TABLE 2

| | Concentration | Mixture | Type of sugar/compound |
|---|---|---|---|
| Xylose | g/kg | 33.9 | C5 sugar |
| Arabinose | g/kg | 3.4 | C5 sugar |
| Glucose | g/kg | 6.8 | C6 sugar |
| Galactose | g/kg | 1.1 | C6 sugar |
| Mannose | g/kg | 0.4 | C6 sugar |
| 5-HMF | g/kg | 0.4 | Potential fermentation inhibitor |
| | | | according to the concentration |
| Furfural | g/kg | 0.2 | Potential fermentation inhibitor |
| | | | according to the concentration |
| Acetic acid | g/kg | 1.2 | Potential fermentation inhibitor |
| | | | according to the concentration |

At the beginning of the reaction, the reaction medium contains:
8.3 kg of C6 sugars (glucose+galactose+mannose),
37.3 kg of C5 sugars (xylose+arabinose),
0.4 kg of 5-HMF,
0.2 kg of furfural.

The way the fermentation is carried out is described in detail below:

The fermentation lasted 6 hours and shows that the glucose, the galactose and the mannose (C6 sugars, in dashed lines) are completely consumed. The arabinose and the xylose (C5 sugars) are not consumed. The furfural and the 5-HMF were consumed by the yeast which detoxifies its medium, to produce furfuryl alcohol and 5-(hydroxymethyl)furfuryl alcohol (also known as 2,5-bis(hydroxymethyl)furan) which do not inhibit the yeast.

It has thus been found that the initial liquor L0, composed of 45.6 kg of monomeric sugars (18% C6 sugars/82% C5 sugars), is purified in a few hours to give a liquor L1 composed of 37.3 kg of monomeric sugars (0% C6 sugars and 100% C5 sugars).

Under anaerobic conditions, at 33° C. and at pH 5.3, the yeasts consume the C6 sugars (predominantly to produce ethanol) and the inhibitors (5-HMF and furfural) in six hours.

At the end of the reaction, the medium contains a liquor L1, the composition of which is given in table 2 below, with concentrations expressed in g per kg of medium:

TABLE 3

| | Concen- | | |
|---|---|---|---|
| | tration | Liquor L1 | Type of sugar/compound |
| Xylose | g/kg | 33.9 | C5 sugar |
| Arabinose | g/kg | 3.4 | C5 sugar |
| Glucose | g/kg | 0 | C6 sugar |
| Galactose | g/kg | 0 | C6 sugar |
| Mannose | g/kg | 0 | C6 sugar |
| 5-HMF | g/kg | 0 | Potential fermentation inhibitor |
| | | | according to the concentration |
| Furfural | g/kg | 0 | Potential fermentation inhibitor |
| | | | according to the concentration |
| Acetic acid | g/kg | 1.2 | Potential fermentation inhibitor |
| | | | according to the concentration |
| 5-(Hydroxy- | g/kg | 0.4 | Product of the |
| methyl)furfuryl | | | consumption of the 5-HMF |
| alcohol | | | |
| Furfuryl | g/kg | 0.2 | Product of the |
| alcohol | | | consumption of the furfural |
| Ethanol | g/kg | 2.9 | Product of the |
| | | | consumption of the C6 sugars |

There is thus present, after 6 hours of reaction:
0 kg of C6 sugars (glucose+galactose+mannose), i.e. a consumption of 100% of the C6 sugars,
37.3 kg of C5 sugars (xylose+arabinose), i.e. a consumption of only 1.6% of the C5 sugars,
0 kg of 5-HMF, i.e. a consumption of 100% of the 5-HMF,
0 kg of furfural, i.e. a consumption of 100% of the furfural,
0.4 kg of 5-(hydroxymethyl)furfuryl alcohol,
0.2 kg of furfuryl alcohol,
2.9 kg of ethanol, i.e. an ethanol/C6 sugars yield of 0.35 kgₑₜₕₐₙₒₗ/kg_{C6 sugars}.

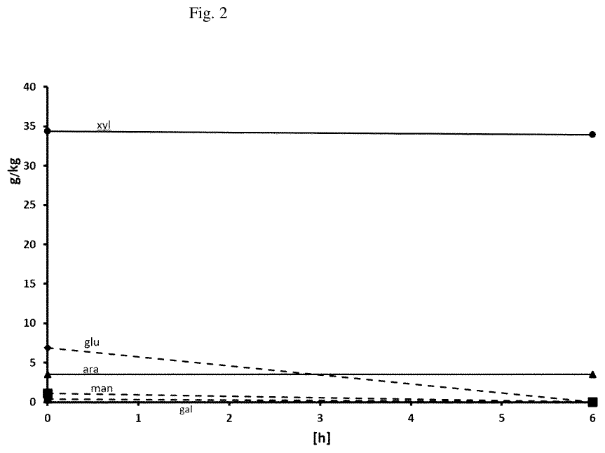

The data in table 2 and the graph in FIG. 2 confirm that the yeast has no action on C5 sugars, the content of which remains constant. On the other hand, the yeast has consumed both all of the C6 sugars and the two inhibitors, to convert them at least in part into alcohols (it being possible for another part to be consumed by the yeast for its own growth). The treatment by the yeast thus gives spectacular results, with a liquor purified in C5 sugars which no longer contains C6 sugars at all and the inhibitors of which have been, simultaneously, completely removed as well.

The fact that a part at least of the C6 sugars has been converted into ethanol is also very advantageous because ethanol is a product which is highly upgradable and which can be produced in processes treating lignocellulosic biomass.

It is also observed, from the data of the graph in FIG. 2, that the duration of the fermentation for the C6 sugars to be completely consumed is 6 hours, which is a reasonable period of time, which can be further reduced, by varying in particular the amount of yeast added, or if reduced but nonzero contents of C6 sugars are acceptable. Experiments have thus been successfully carried out with much shorter fermentation times, in particular from 30 minutes to 2 hours, for example a duration of approximately 1 hour.

FIG. 3 represents an alternative form of FIG. 1: starting from the same liquor L0 as in FIG. 1 and as in example 1, a fermentation is carried out, this time under aerobic conditions, of the liquor L0 with the same microorganism as in example 2, the yeast Saccharomyces cerevisiae, in a native version which is not genetically modified and which exhibits the distinguishing feature of selectively consuming the C6 sugars and not the C5 sugars.

The reactions which result from the action of the yeast are shown diagrammatically in FIG. 3, which represents the bioreactor 1, with the following inputs:
2: the yeast,
3: a base (for regulating the pH and keeping the reaction medium under the conditions necessary for the activity of the chosen yeast), of NH₄OH, KOH or NaOH type, for example,
4: nutrients for the yeast,
5: the liquor L0, which contains, in the aqueous phase, C5 sugars 51, C6 sugars 52, 5-HMF 53, furfural 54 and acetic acid 60,
and with the exiting products:
6: emission of CO₂ in the gas phase, discharged at the top part of the bioreactor, evidence of the activity of the yeast,
5′: withdrawal of a liquor L1, resulting from the liquor L0 after the action of the yeast on the liquor L0, and which contains the propagated yeast 2, C5 sugars 51, 5-(hydroxymethyl)furfuryl alcohol 55 and furfuryl alcohol 56,
7: a feed of air to be under aerobic conditions.

It has been verified that the yeast thus has no action on the C5 sugars, the content of which remains constant. On the other hand, the yeast has consumed both all of the C6 sugars and the two inhibitors (5-HMF and furfural), and also the acetic acid, to convert them at least in part into alcohols (it being possible for another part to be consumed by the yeast for its own growth). The treatment by the yeast thus gives spectacular results, with a liquor purified in C5 sugars which no longer contains C6 sugars at all and the inhibitors of which have been, simultaneously, completely removed as well, while multiplying the yeast.

In conclusion, with regard to this purification stage, it is seen that success is achieved in purifying mixtures of sugars in order to obtain liquors containing the type of sugars which it is desired to retain (C5 sugars), by choosing a selective microorganism, which can also, optionally, also consume/remove undesirable compounds/impurities, and in particular inhibitors of biochemical reactions, such as furan derivatives, in the mixtures of sugars. Its implementation is simple, in one stage of aerobic fermentation, and its effectiveness formidable, with optionally complete removals of the compounds which it is desired to remove from sugary liquors.

It makes it possible to obtain very pure C5 sugary liquors, with no or a very low content of C6 sugars, with a purity which had not previously been possible to achieve by separation methods, of the type of selective adsorption on zeolites, as described in the patents cited in the preamble to the present patent application.

It is in addition highly advantageous, in that it results not really in carrying out a separation between two types of sugars but in obtaining a very pure type of sugar, and in converting the other sugar (C6 sugar) into an upgradable product, such as ethanol. With the combination of the C5 sugars and of the ethanol resulting from the conversion of the C6 sugars which is obtained with the process according to the invention, several possibilities present themselves, in particular according to the upgrading routes envisaged and the proportion between the two types of sugars in the initial sugary liquor. Thus, it is subsequently possible to separate the C5 sugars from the ethanol and to obtain, on the one hand, very pure C5 sugars and, on the other hand, ethanol, which products can be upgraded/used separately. It is also possible to keep them together for a common upgrading. It is further possible to retain a low content of ethanol with the C5 sugars, to benefit from the preservative/antimicrobial effect of the alcohol on the sugars.

Processes for the Conversion of Lignocellulosic Biomass Producing Sugary Liquors and Incorporating the Stage of Purification of Said Sugary Liquors which is Described Above

Three conversion processes are described below:
a process A for conversion into alcohol (ethanol),
a process B for conversion into sugar(s), and
a “hybrid” process C, targeted at obtaining both an alcohol and sugar(s).
   Process A for Conversion of Biomass into Ethanol

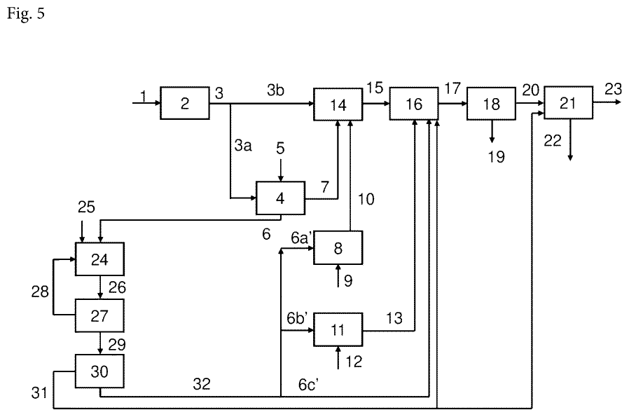

This is illustrated with the help of FIGS. 4 and 5.

FIG. 4 describes a conversion process without the purification stage described above. The references in the figures represent the following streams/devices/stages:
1: Biomass
2: Conditioning and pretreatment
3: Pretreated biomass
3a: Pretreated biomass to the filtration tool
3b: Pretreated biomass to enzymatic hydrolysis (optional)
4: Solid/liquid separation (a belt filter or a filter press, for example) to extract a sugary liquor, with an optional washing stage
5: Water as contacting and/or washing fluid
6: Sugary liquor containing predominantly C5 sugars
6a: Sugary liquor comprising predominantly C5 sugars to the production of enzymes
6b: Sugary liquor comprising predominantly C5 sugars to the propagation of yeasts
7: Pretreated and washed biomass
8: Reactor for the production of enzymes
9: Fungal inoculum (for example Trichoderma reesei) for the production of enzymes
10: Enzyme cocktail (mixture of one or more enzymes, containing in particular cellulases, β-glucosidases, xylanases, and the like)
11: Reactor for the propagation of yeasts
12: Yeasts
13: Propagated yeasts
14: Enzymatic hydrolysis reactor
15: Hydrolysate
16: Fermentation reactor
17: Fermentation must (wine)
18: Solid/liquid separation (for example a filter press) with an optional stage of washing the solid residue with a solvent (for example water)
19: Solid residue (composed predominantly of lignin)
20: Liquid product of the filtration, in this instance filtered wine
21: Separation of the ethanol and of the water (distillation column, sieve, for example)
22: Liquid residue (vinasses)
23: Purified ethanol

Throughout the present text, and in particular in the description of the figures, the term of reactor does not require that there be only a single one of them, and can be understood as a unit comprising at least one reactor. It is the same for all the other devices, in particular the tools of filtration, solid/liquid separation, distillation column, and the like, type (for the sake of brevity).

If the sequence of events of the process according to FIG. 4 is taken: The biomass 1 undergoes a conditioning/pretreatment in the unit 2 (optional mechanical grinding, dust removal, removal of possible metal parts, then, in this instance by way of example, impregnation with an acidic liquor and cooking/steam explosion of the pretreated biomass 3). A part of this pretreated biomass, the stream 3a, leaves to a solid/liquid filtration tool 4, said tool being also fed with water 5 as contacting and/or washing water. As a first step, the pretreated biomass is contacted with water to improve its filterability. The mixture is subsequently filtered on a belt filter and washed with water. Another part of the pretreated biomass, the stream 3b, can be sent to the enzymatic hydrolysis reactor 14. This reactor 14 is also fed with the stream 7, which is the solid fraction resulting from the filtration in the unit 4, which is thus the washed solid pretreated biomass.

It should be noted that, throughout the present text, “solid” is to be understood in opposition to “liquid” but that any solid fraction can still contain a certain proportion of liquid (and vice versa). This proportion of liquid in a solid fraction can be evaluated by measurement of its dry matter (DM) content, which is measured according to the standard ASTM E1756-08 (2015), “Standard Test Method for Determination of Total Solids in Biomass”.

At the outlet of the reactor 14, there is available a stream 15 of hydrolysate (sugars) which subsequently feeds a fermentation reactor 16 in order to be converted into ethanol: at the outlet of the fermentation reactor 16, a fermentation must, also known as wine, which contains ethanol in water, is obtained. A solid/liquid separation tool 18 will separate a solid ligneous residue 19 from a liquid fraction 20, the filtered wine. This filtered wine is conveyed into a separation tool 21 (distillation column) in order to obtain ethanol 23 and an aqueous liquid residue (known as vinasse).

At the outlet of the separation tool 4, the liquid fraction 6 is an aqueous sugary liquor comprising predominantly C5 sugars. All or part of this fraction, the stream 6a, can proceed to the reactor 8 for the production of enzymes, which produces an enzymatic cocktail 10 to feed the enzymatic hydrolysis reactor 14: this stream can thus constitute a substrate for growth and/or production with regard to the microorganisms of Trichoderma reesei type which produce this mixture of enzymes 10. All or some of this stream 6, the stream 6b, can also proceed to the reactor 11 for propagation of the yeasts 13 which feed the fermentation reactor 16. Here again, the sugary liquor 6b can act as propagation substrate with regard to the yeasts, for example Saccharomyces, in particular the species Saccharomyces cerevisiae.

It should furthermore be noted that the enzymatic hydrolysis operation 14 is preferably carried out in two stages: liquefaction with a fed-batch of substrate and then enzymatic hydrolysis in batch mode (optionally with two reactors in series, or in the same reactor), in order to work at the highest possible DM content and to convert lignocellulosic substrates for which the rheology may be complex. A neutralization of the medium can be carried out before the enzymatic hydrolysis, for example by addition of a basic solution in the case where the pretreatment is carried out under acidic conditions.

It should also be noted that the enzymatic hydrolysis and the fermentation can be carried out simultaneously (SSCF). When they are done one after the other, an adjustment of the pH of the medium between the units 14 and 16 can be carried out.

It should also be noted that the production of enzymes and/or the propagation of yeasts can take place on the site of treatment of the biomass, as represented in FIG. 4. However, one or the other can also be carried out ex situ: ready-for-use enzymes or yeasts can thus be brought on site. This explains why, depending on the choices made in this respect, the stream can be divided into two streams 6a, 6b (with a distribution between the two streams which may or may not be equal, depending on the requirements) or be used only for the propagation of the yeasts or only for the production of the enzymes.

FIG. 5 represents the process of FIG. 4 but with the modifications according to the invention and the following additional references:
6a′: Sugary liquor comprising solely C5 sugars to the production of enzymes
6b′: Sugary liquor comprising solely C5 sugars to the propagation of yeasts
6c′: Sugary liquor comprising solely C5 sugars to the fermentation
24: Biochemical purification reactor
25: Yeasts consuming the C6 sugars
26: Sugary liquor comprising solely C5 sugars, the yeasts and ethanol
27: Solid/liquid separation (a centrifuge, a filter or ultrafiltration unit, for example)
28: Recycled yeasts consuming the C6 sugars
29: Sugary liquor comprising solely C5 sugars and optionally ethanol
30: Separation of the ethanol and of the liquor of C5 sugars (a stripper or an evaporator, for example), optional stage
31: stripped ethanol stream
32: Sugary liquor comprising solely C5 sugars

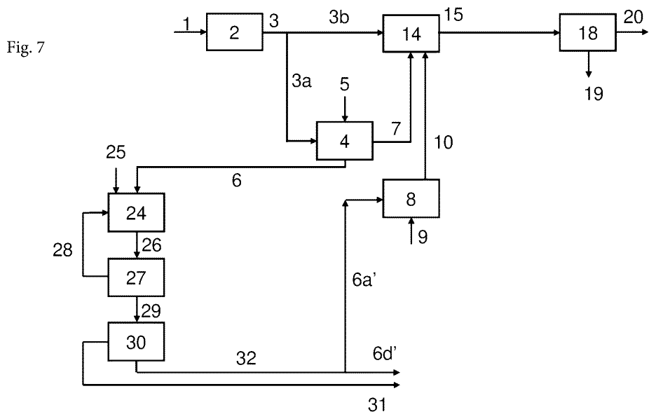

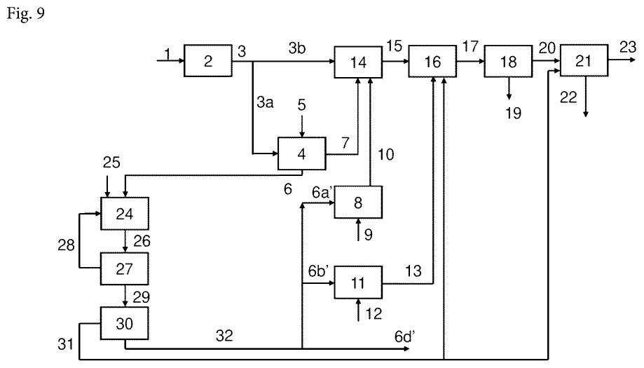

It should be noted that the stream 3 of pretreated biomass is divided in FIG. 5 into two streams: the stream 3a, which leaves to the tool 4, and the stream 3b, which leaves directly to the enzymatic hydrolysis reactor 14. However, here, as in the case of the processes represented in the following FIGS. 7 and 9, the relative proportion between the streams 3a and 3b is variable.

Also, according to one embodiment of the invention, it is also foreseen for the entire pretreated biomass stream 3 to pass first through the tool 4 before the solid portion is separated and sent to the hydrolysis reactor 14: there is only one stream 3/3a entering in its entirety the tool 4 and there is no stream 3b: the reactor 14 is no longer fed with stream 3b. This embodiment is more particularly recommended in the case of the production of sugar (FIG. 7), more than in the case of the production of alcohol (FIG. 5) or of sugar and of alcohol (FIG. 9).

This scenario having no stream 3b thus also comes within the invention, whether it concerns a process for the production of alcohol, of sugar or alcohol/sugar hybrid.

The stream rich in ethanol 31 can be introduced separately or as a mixture with the filtered wine at the inlet of the section 21 for separation of the ethanol and of the water, or into the fermenter 16. The operating conditions of the stripper can be adjusted so as to produce a stream rich in C5 sugars and a stream rich in ethanol.

The ethanol produced 31 can also be recycled to the pretreatment stage 2 or to the stage of filtration and washing of biomass 4.

The stream 32 of sugary liquor comprising solely C5 sugars can be used as substrate for the production of enzymes, the propagation of the yeasts or in fermentation.

In this instance, the stream 6, which contains more C5 sugars than C6 sugars, is purified in the following way: it is introduced into a biochemical purification reactor 24, which can include a preliminary stage of neutralization of the liquor 6, for example with a basic solution of sodium or potassium hydroxide, in the case where the biomass has been pretreated under acidic conditions. The reactor 24 is also fed with microorganisms 25 (yeasts in particular) which exhibit the distinguishing feature of consuming the C6 sugars only. At the outlet of the reactor 24, there is available a stream 26 of sugary liquor depleted in C6 sugars. The solid/liquid separation tool 27 makes it possible to recover a solid fraction 28, which comprises the insoluble yeasts, which will be able, in all or part, to be recycled in the reactor 24. The separated liquid fraction 29 is thus a C5 sugary liquor, without microorganisms and with less/no C6 sugars and possibly ethanol, which is a product of the conversion of the C6 sugars by the yeasts. This fraction 29 is sent to a separation tool 30 which separates the sugars from the ethanol when ethanol is present in the fraction 29 in a measurable content. Then the separated sugary liquor 32 can be used in different ways: all or part in the form of a stream 6a′ to the production 8 of enzymes, all or part of a stream 6b′ to the propagation of the yeasts for the fermentation 11 of biomass (as in the process of FIG. 4) and all or part of a stream 6c′ can be sent to the fermentation reactor 16. The ethanol stream 31 can, for its part, be sent to the fermentation reactor 16 (either during the fermentation, or be added to the stream exiting the reactor 16) or also be added to the stream 20 at the inlet of the ethanol separation device 21. The relative proportions of the streams 6a′, 6b′ and 6c′ are adjustable, in particular if the propagation of the yeasts and/or the production of enzymes are carried out in situ or ex situ (in this case, the streams 6a′ and/or 6b′ do not exist). In all cases, an additional proportion of ethanol 6c′ is added to the ethanol production exiting the fermenter 16, at the fermenter 16 or downstream of the latter (the terms “upstream” and “downstream” are understood in the present text according to the general direction of biomass through the installation).

Incorporating the biochemical purification of the mixture of sugars 6 makes it possible, in the end, to increase the amount of ethanol produced for a given amount of biomass, and thus to increase its conversion yield.

Process B for Conversion of Biomass into Sugars

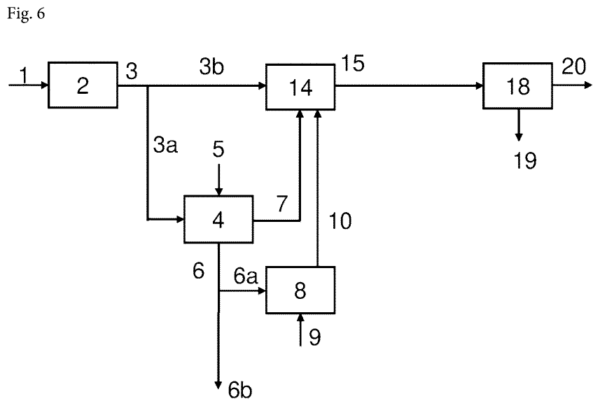

It is illustrated by FIGS. 6 and 7.

FIG. 6 represents the process without the purification according to the invention. It is a matter in this instance of halting the conversion of biomass at the stage of the production of sugars, thus without fermentation. In comparison with FIG. 4, the sugary liquor 20, predominantly consisting of C6 sugars, is thus the first final product which is upgraded, the stream 6a can be used to act as substrate for the growth and/or the production of enzymes in the reactor 8 (if enzymes are produced in situ) and the stream 6b is the second final product which is can be upgraded, a liquor of predominantly C5 sugars but which still contains C6 sugars.

FIG. 7 is a process which incorporates, in the process of FIG. 6, the purification of sugary liquor according to the invention: The reactors/tools 24, 27 and 30 of the process of FIG. 5, which are operated in the same way, are re-encountered therein. In this instance, the sugary liquor 6 containing predominantly C5 sugars is, after purification and separation of the yeasts, purified to give a liquor 32, which is divided in part into a stream 6a′ of C5 sugars, in order to act as substrate for the growth/production of enzymes in the reactor 8, if the production of enzymes takes place in situ, and in all or part into a stream 6d′, which is a purified C5 sugary liquor which can be upgraded as is. This liquor of C5 sugars, referenced 6d′, is very pure, purer than the liquor of the stream 6b of the process of FIG. 6; it can thus be used as is.

“Hybrid” Process C for Conversion of Biomass into Ethanol and into Sugar(s)

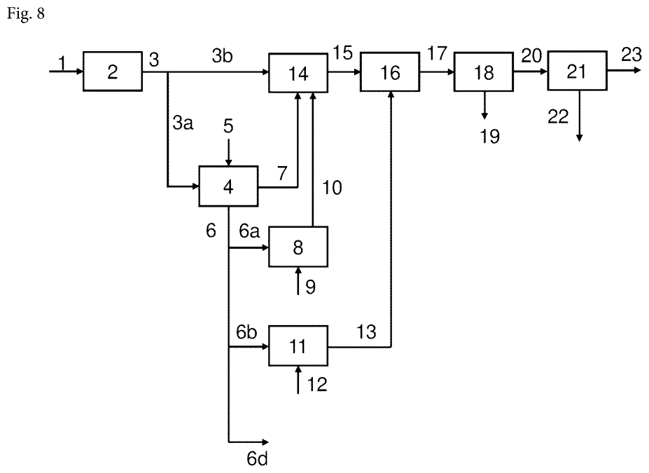

It is illustrated by FIGS. 8 and 9.

The process according to FIG. 8 does not use the purification according to the invention. It is a matter in this instance of producing sugars and ethanol in parallel. In comparison with the process of FIG. 4, which is targeted at producing ethanol, in this instance there is available both:
production of ethanol, which is the stream 23 obtained as in the process of FIG. 4, and
production of predominantly C5 sugars: this is the stream 6d, resulting from the stream 6 as in the process in FIG. 4. This stream 6d can be part of the stream 6, or all of this stream (if enzymes are produced ex situ, and/or yeasts are propagated ex situ, or also if the use of other, external, sources is preferred for the growth/propagation of the microorganisms/production of enzymes).

The process of FIG. 9 incorporates, in the process of FIG. 8, the purification according to the invention: The reactors/tools 24, 27 and 30 described above, the obtaining of a stream 32 purified in C5 sugars and that of a stream 6d′ which constitutes all or part of the liquor 32 and which constitutes a sugary liquor purified in C5 sugars which can be upgraded as is, in parallel with the production of ethanol 23, are re-encountered. The ethanol stream 31 resulting from the separation 30 can, as in the case of the process of FIG. 5, be reinjected into the fermenter 16, or downstream of the latter, as a mixture with the stream exiting the fermenter 16, or also upstream of the device 21 (distillation column) separating the ethanol from the water. A doubly beneficial situation thus exists in this instance, with both an improved ethanol yield and an improved quality/purity of liquor of C5 sugars.

From these various embodiments of the invention, all the advantages thereof are seen:

The hydrolysate of lignocellulosic sugars composed of a mixture of sugars having 6 and 5 carbon atoms is purified to give a hydrolysate containing only sugars having 5 carbon atoms:

The use of this “pure” mixture of C5 sugars is beneficial for many chemical conversion applications (conversion of xylose to xylitol by catalysis) or biochemical conversion applications (propagation of yeasts, induction of fungus for the production of enzymes, for example).

The complex and expensive physical separation of C5 and C6 sugars (for example by nanofiltration, membranes, ion-exchange resins, zeolites, and the like) is thus avoided.

The hydrolysate of lignocellulosic sugars composed of a mixture of sugars having 6 and 5 carbon atoms is purified to give a hydrolysate containing only sugars having 5 carbon atoms but also containing fewer/more compounds described as inhibitors, such as furfural or 5-(hydroxymethyl)furfural (5-HMF): These two compounds are, for example, inhibitors for fermentative reactions.

In the case of the use of the yeast S. cerevisiae and under anaerobic conditions in order to proceed to the biochemical purification of the mixture of sugars, the consumption of the C6 sugars produces ethanol, which can be recovered and incorporated in the biomass conversion process.

In the case of the use of the yeast S. cerevisiae and under aerobic conditions in order to proceed to the biochemical purification of the mixture of sugars, the consumption of the C6 sugars produces additional microorganisms (multiplication of the yeasts), which can be recovered and incorporated in the process for the conversion of biomass.

Under aerobic conditions, the yeast S. cerevisiae can also consume the acetic acid of the medium, which is also an inhibitor of fermentative reactions or of the growth of the fungus T. reesei.

The biochemical purification is carried out on a clear medium; it is easy to separate the microorganisms by a solid/liquid separation, for example a centrifugation or ultrafiltration membranes, in order to remove and recycle the microorganisms.

The ethanol produced can advantageously be used in the process for the production of 2G sugars:
a fraction of the ethanol can be used in the pretreatment 2,
a fraction of the ethanol can also or alternatively be used as washing fluid for the filtration stage 4,
a fraction of the ethanol can also or alternatively be used as fluid for washing the unconverted residual solid residue. This makes it possible to increase the NCV (acronym for net calorific value) of the solid residue which can be incinerated in a cogeneration boiler for the production of electricity and steam, or to extract therefrom a soluble fraction for better upgrading (fuel base, or for bioproducts),
a fraction of the ethanol can also or alternatively be incorporated in the reaction medium of the enzymatic hydrolysis reactor 14, which makes it possible to limit the risks of contamination.

The stage of separation 30 of the purified sugars and of the ethanol is carried out so as to concentrate the sugars up to a content of, for example, 50 g/kg, which is all the more advantageous since this sugary liquor is used for the production of the enzymes and propagation of the yeasts. The purification stages are thus perfectly integrated into the production of ethanol and/or of 2G sugars.

### Examples Employing a Biomass Conversion Process

Example 3 According to the Process A for Conversion of Biomass into Ethanol

It is carried out according to the process of FIG. 5, in comparison with that of FIG. 4.

In this example, the reactions are carried out in SSCF, stages 14 (enzymatic hydrolysis) and 16 (ethanolic fermentation) are carried out in one and the same reactor and are carried out at a temperature of 33° C. and pH 5.3 under anaerobic conditions. In this example according to the invention, stage 30 of separation of the ethanol from the C5 liquor is not carried out. In the scheme in accordance with the invention, the purification in stage 24 makes it possible to reduce the concentration of 5-HMF (changing from 2.80 to 0.56 g/l) and of furfural (changing from 1.60 to 0.32 g/l) present at the start of the reaction. This purification also produces ethanol, from the C6 sugars (2.84 g/l).

As these compounds are inhibitors for the yeast Saccharomyces cerevisiae, ethanol production undergoes latency, indeed even an inhibition, of the fermentative activity in the process of FIG. 4 without the purification of the sugars: specifically, the fermentative reaction time is prolonged, or the yeasts may even not ferment.

After 144 hours of reaction, the ethanolic titer of the ethanol produced according to the process of FIG. 5 according to the invention is 52.4 g/l compared with 49.3 g/l for the process of FIG. 4.

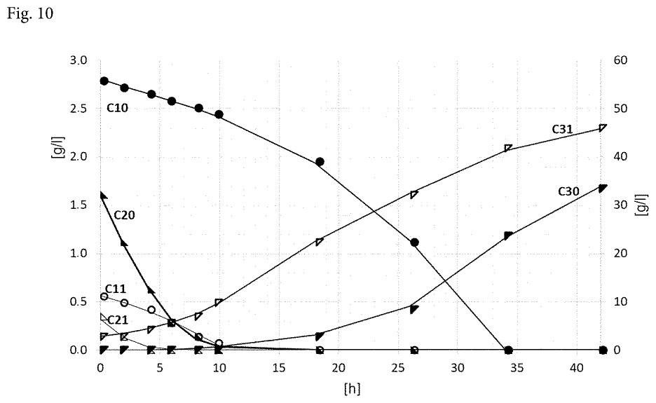

The difference in ethanolic titer is even greater before 50 hours of reaction. For example, after 42 hours of fermentation, the invention makes it possible to change from 33.8 to 45.9 g/l of ethanol. It is this which can be observed from the graph of FIG. 10, which represents the changes in concentration in g/l of different compounds during the SSCF (simultaneous enzymatic hydrolysis and fermentation) reaction:
curve C10: change in the 5-HMF content by applying the process of FIG. 4 (without purification)
curve C11: change in the 5-HMF content by applying the process of FIG. 5 (with purification)
curve C20: change in the furfural content by applying the process of FIG. 4 (without purification)
curve C21: change in the furfural content by applying the process of FIG. 5 (with purification)
curve C30: change in the ethanol content by applying the process of FIG. 4 (without purification)
curve C31: change in the ethanol content by applying the process of FIG. 5 (with purification).

It is seen, from this graph, that, at t=0, the purification of the C5 sugars made it possible to reduce the load of inhibitor: furfural, 5-HMF, and that, after 50 hours of reaction, the ethanol productivity is much higher with the purification according to the invention. At the end of the reaction, the amount of ethanol produced is greater.

In addition, this increase in the ethanol concentration makes it possible to reduce the energy consumption necessary for the stage of distillation in the column 21, to separate the ethanol from the water.

When a reaction time of 144 hours is chosen, the energy gain is 4.5%. When a reaction time of 42 hours is chosen, the energy gain is 21%.

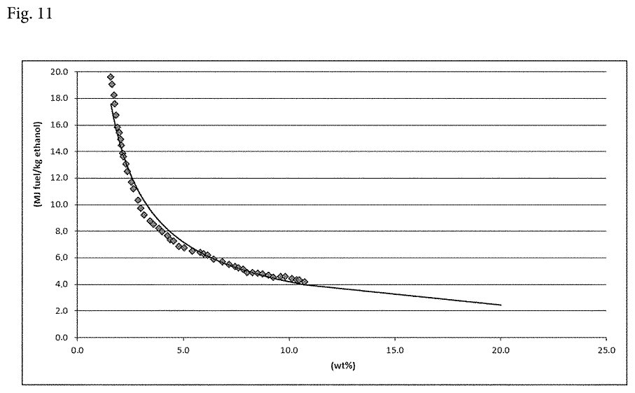

This is what is shown in FIG. 11, which represents the energy necessary (in MJ) per kg of ethanol present in the feedstock of the distillation column as a function of the ethanol concentration of the solution to be separated, in order to recover, at the distillation top, a rich stream having 94% by weight of ethanol and, at the column bottom, a stream containing 0.02% by weight of ethanol, and which results from the publication by Vane, L. M., “Separation Technologies for the Recovery and Dehydration of Alcohols from Fermentation Broths”, Biofuels, Bioproducts and Biorefining, Society of Chemical Industry, London, UK, 2(6):553-588 (2008).

1. A process for the treatment of a lignocellulosic biomass, said process comprising:
a) a stage of pretreatment (2) of biomass, comprising a cooking of biomass, optionally accompanied by steam explosion, in order to obtain a pretreated biomass (3),
b) a first stage of solid/liquid separation of all or part (3a) of the pretreated biomass (3) obtained in stage a) into a first solid fraction (7) of pretreated biomass and a first liquid fraction (6) comprising a mixture of compounds, in particular in the aqueous phase, comprising C5 sugars with 5 carbons and C6 sugars with 6 carbons, and the content by weight of C5 sugars of which is greater than the content by weight of C6 sugars,
c) a stage of enzymatic hydrolysis (14) of the first solid fraction (7) of pretreated biomass obtained in stage b), in order to obtain a hydrolysate (15) in the form of sugar(s), including C6 sugars with 6 carbons,
d) a stage of purification (24) of said first liquid fraction obtained in stage b) by bringing said first liquid fraction (6) into contact with first microorganisms consuming, among the sugars of said fraction, essentially only the C6 sugars, in order to obtain a purified liquid fraction (26) containing C5 sugars and depleted in C6 sugars,
e) a second stage of solid/liquid separation (27) of the purified liquid fraction (26) containing C5 sugars and depleted in C6 sugars obtained in stage d) in order to obtain a second solid fraction (28) comprising the first microorganisms and a second liquid fraction (29) containing C5 sugars and depleted in C6 sugars.

2. The process as claimed in claim 1, wherein only a first part (3a) of the pretreated biomass (3) obtained in stage a) is conveyed to the first stage b) of solid/liquid separation, and in that a second part (3b) of the pretreated biomass (3) is conveyed directly to the enzymatic hydrolysis stage c).

3. The process as claimed in claim 1, further comprising a stage f) of fermentation (16) by third microorganisms of the hydrolysate in the form of sugar(s) obtained in stage c), in order to obtain a fermented biomass (17) comprising at least one alcohol, in particular ethanol.

4. The process as claimed claim 3, wherein the enzymatic hydrolysis stage c) and the fermentation stage f) are carried out simultaneously.

5. The process as claimed in claim 1, wherein at least a part of the second solid fraction (28) comprising the first microorganisms which are obtained in stage e) is recycled in the purification stage d) (24).

6. The process as claimed in claim 1, wherein the second liquid fraction (29) containing C5 sugars and depleted in C6 sugars which is obtained in stage e) contains alcohol, in particular ethanol, obtained by conversion of all or part of the C6 sugars under the action of the first microorganisms, and in that, in a stage g), in particular by evaporation or stripping, said second liquid fraction is separated (30) into a third liquid fraction (32) enriched in sugars and a fourth liquid fraction (31) enriched in alcohol.

7. The process as claimed in claim 6, wherein all or part of the fourth liquid fraction (31) containing the alcohol obtained in stage g) is sent to the fermentation stage f) (16) or in that all or part of said fourth liquid fraction is mixed with the fermented biomass (17) comprising at least one alcohol obtained on conclusion of the fermentation stage f).

8. The process as claimed in claim 1, further comprising a stage h) of production of enzymes (8) from second microorganisms, in particular fungi, in order to use said enzymes to ensure the enzymatic hydrolysis of stage c), and in that all or part (6a′) of the second liquid fraction (29) containing C5 sugars and depleted in C6 sugars which is obtained in stage e) or the third liquid fraction (32) enriched in sugars which is obtained in stage g) is sent to said stage h) as substrate for growth of the second microorganisms and/or for production of the enzymes.

9. The process as claimed in claim 3, further comprising a stage i) of propagation (11) of third fermentation microorganisms, in order to use said microorganisms, in particular yeasts, to ensure the fermentation of stage f), and in that all or part (6b′) of the second liquid fraction (29) containing C5 sugars and depleted in C6 sugars which is obtained in stage e) or the third liquid fraction (32) enriched in sugars which is obtained in stage g) is sent to said stage i) as substrate for the propagation of said third microorganisms.

10. The process as claimed in claim 1, wherein all or part (6c′) of the second liquid fraction (29) containing C5 sugars and depleted in C6 sugars which is obtained in stage e) or the third liquid fraction (32) enriched in sugars which is obtained in stage g) is sent to the fermentation stage f) (16).

11. The process as claimed in claim 1, wherein the first liquid fraction (6) obtained in stage b) also comprises furfural, and in that the first microorganisms also consume furfural, in particular at least in part by conversion of the furfural into alcohol, in particular into furfuryl alcohol.

12. The process as claimed in claim 1, wherein the first liquid fraction (6) obtained in stage b) also comprises 5-(hydroxymethyl)furfural 5-HMF, and in that the first microorganisms also consume 5-(hydroxymethyl)furfural, in particular at least in part by conversion of the 5-(hydroxymethyl)furfural into 5-(hydroxymethyl)furfuryl alcohol.

13. The process as claimed in claim 1, wherein the first microorganisms used in stage d) and optionally the third microorganisms used in stage i) are chosen from yeasts, bacteria and fungi.

14. The process as claimed in claim 8, wherein the second microorganisms are chosen from fungi.

15. The process as claimed in claim 1, wherein the pretreatment (2) according to stage a) of biomass comprises:
a1) a substage of impregnation of biomass (1) with a liquor, in particular an acidic liquor, in order to obtain an impregnated biomass,
a2) a substage of cooking the impregnated biomass, optionally accompanied by steam explosion, in order to obtain a pretreated biomass (3).

16. The process as claimed in claim 3, further comprising:
a stage j) of separation, in particular by distillation (21), of the fermented biomass obtained in stage f) in a form of alcohol (20), with an optional stage k) of solid/liquid separation of the fermented biomass before or after said separation stage j).

17. An installation for the treatment of a lignocellulosic biomass, said installation comprising:
a) a unit for the pretreatment (2) of biomass, comprising a device for cooking biomass, optionally accompanied by steam explosion, in order to obtain a pretreated biomass,
b) a first unit for solid/liquid separation (4), comprising in particular a filtration device, of all or part (3a) of the pretreated biomass (3) obtained in the unit a) into a first solid fraction (7) of pretreated biomass and a first liquid fraction (6) comprising a mixture of compounds, in particular in the aqueous phase, comprising C5 sugars with 5 carbons and C6 sugars with 6 carbons, and the content by weight of C5 sugars of which is greater than the content by weight of C6 sugars,
c) a unit for enzymatic hydrolysis (14) of the first solid fraction (7) of pretreated biomass obtained in stage b), in order to obtain a hydrolysate (15) in the form of sugar(s), including C6 sugars with 6 carbons,
a unit d) for purification (24) of said first liquid fraction (6) with said first liquid fraction being brought into contact with first microorganisms consuming, among the sugars of said fraction, essentially only the C6 sugars, in order to obtain a purified liquid fraction (26) containing C5 sugars and depleted in C6 sugars, and
a second unit e) for solid/liquid separation (27), comprising in particular at least one filtration or centrifugation device, of the purified liquid fraction (26) containing C5 sugars and depleted in C6 sugars obtained in the unit d) in order to obtain a second solid fraction (28) comprising the first microorganisms and a second liquid fraction (29) containing C5 sugars and depleted in C6 sugars.

18. The installation as claimed in claim 17, wherein the second liquid fraction (29) containing C5 sugars and depleted in C6 sugars which is obtained in the unit e) contains alcohol, in particular ethanol, obtained by conversion of all or part of the C6 sugars under the action of the first microorganisms, and in that said installation comprises a unit g) for separation (30) of said second liquid fraction into a third liquid fraction (32) enriched in sugars and a fourth liquid fraction (31) enriched in alcohol, said separation unit g) comprising at least one evaporation or stripping device.

19. The process as claimed in claim 1, wherein the first microorganisms used in stage d) and optionally the third microorganisms used in stage i) are chosen from yeasts in the genus Saccharomyces and bacteria in the genus Corynebacterium.

20. The process as claimed in claim 8, wherein the second microorganisms are chosen from filamentous fungi.
